(19) United States
(12) Patent Application Publication
(10) Pub. No.: US 2015/0327798 A1
(43) Pub. Date: Nov. 19, 2015

(54) DEVICE AND METHODS FOR THE TREATMENT OF HEARING CONDITIONS

(71) Applicant: William H. Maxwell, South Portland, ME (US)

(72) Inventor: William H. Maxwell, South Portland, ME (US)

(21) Appl. No.: 14/715,466

(22) Filed: May 18, 2015

Related U.S. Application Data
(60) Provisional application No. 61/994,932, filed on May 18, 2014.

(51) Int. Cl. A61B 5/12; H04R 25/00

(52) U.S. Cl. CPC A61B 5/125; H04R 25/70

(57) ABSTRACT

A device and method for the diagnosis and/or treatment of any one or more of hearing loss, tinnitus and hyperacusis. The device is configured with programming to enable a patient to select from one or more program blocks that establish sound amplification and sound generation functions and to control adjustment of sound amplification and/or generation within a range. A single device of the invention can be used to detect and/or resolve a plurality of conditions including hearing loss, tinnitus and hyperacusis. The acoustic modulator device of the present invention enables effective tinnitus treatment, effective hyperacusis treatment and effective treatment when both exist. The method also includes a graduated process for building patient tolerance to noise amplitude when the patient has hyperacusis including doing so to allow the patient to reach a tinnitus threshold point.

### CROSS REFERENCE TO RELATED APPLICATION

The present application is a nonprovisional and claims the priority benefit of provisional U.S. patent application Ser. No. 61/994,932, filed May 18, 2014, entitled “DEVICE AND METHODS FOR THE TREATMENT OF TINNITUS AND HYPERACUSIS” by the same named inventor. The entire content of that priority application is incorporated herein by reference.

### BACKGROUND OF THE INVENTION

1. Field of the Invention

The present invention relates to the treatment of hearing conditions including hearing loss, tinnitus and hyperacusis. More particularly, the present invention relates to hearing condition treatment methods and devices designed to aid in such treatment methods.

2. Description of the Prior Art

Many people suffer from hearing problems. Those problems can range from inconveniences to debilitating conditions. There are three primary types of hearing problems under consideration—hearing loss, tinnitus and hyperacusis. A person may suffer from one or more of these conditions. The conditions can range from mild to totally disabling and can vary within the range of frequencies that a human can hear. Hearing loss is the most commonly treated hearing condition and the most well-known. It is an inability to hear sounds at certain frequencies or through a spectrum of frequencies. It is often treated with a hearing aid that magnifies sounds entering the ear canal. Tinnitus is more common and without an effective treatment. Hyperacusis is probably equally uncommon and essentially unaddressed.

Tinnitus is a sensation or perception of noise, such as a buzzing, ringing, roaring or whistling, in the absence of a corresponding external sound. There is no way to measure this truly objectively, such as by comparison with noise of known frequency and intensity, as in an audiometric test. The condition is often rated clinically on a simple scale from “minimal” to “disabling” according to the practical difficulties it imposes, such as interference with sleep, quiet activities, and inability to function effectively on a daily basis.

It is generally believed that about 95% of the population experiences some form of tinnitus of which about 80% experiences what professionals characterize as “clinically insignificant tinnitus” and the remaining about 15% experience “clinically significant tinnitus.” For purposes of the present invention, clinically insignificant tinnitus is defined as tinnitus present for less than six months, intermittent and nonpulsatile. Clinically significant tinnitus is defined as tinnitus present for greater than six months, constant and nonpulsatile.

Another hearing condition suffered by individuals is hyperacusis. Hyperacusis is a condition characterized by over-sensitivity to certain sounds. A person with hyperacusis has difficulty tolerating everyday sounds that were previously unnoticed and/or a pan of background “noise.” Examples can include the voices of grandchildren, loud restaurants, sports events, etc. It often affects musicians, making it difficult for them to play in the very loud environment of a rock band or orchestra, which previously gave them no problems. It also makes attendance at loud events difficult for a portion of the population. More importantly, it is more severely disabling at a lesser severity.

Current commonly used methods for treating tinnitus and/or hyperacusis have been largely ineffective. The list of attempted forms of treatment is long, an indication of the difficulty associated with that effort. Treatments may be established patient by patient based on the perceived underlying cause, as difficult as that is to determine. Examples of treatment options include: surgery, neurostimulators, drugs such as melatonin, Zinc supplement, Lignocaine, electrical stimulation, white noise, shaped or filtered noise, auditive stimulation therapy and tinnitus retraining therapy, to name a few. White noise or broadband sound generators are sometimes used to mask aggravating sounds. Hearing condition diagnoses are often inaccurate and current treatment methods are burdensome on the patient while being largely ineffective.

In view of the difficulty in accurately characterizing the underlying cause of these hearing problems and the corresponding difficulty in treating it, there are few patients who achieve satisfactory outcomes under existing treatments. They generally suffer with the condition, particularly those having clinically significant tinnitus, which can lead to a severely debilitating lifestyle. It is noted that the diagnosis and attempted treatment of hearing problems comprise the highest worker disability claim payments. Military healthcare statistics indicate that the number one long-term disability claim for veterans is tinnitus, followed by the loss of limb.

The ineffective treatment options, the increasing population of those more likely to have hearing problems and recent changes in healthcare laws indicate a substantial increase in the number of tinnitus patients requiring treatment. The potentially opposing goals of increasing healthcare coverage and decreasing healthcare costs lead to a need for a more patient-effective way of treating hearing conditions such as hearing loss, tinnitus and hyperacusis. What is needed is a treatment paradigm, a system, a device, a method or any combination thereof to treat hearing conditions including hearing loss, tinnitus and hyperacusis. What is also desired is such a capability that is achieved with a reduction in the burden on the patient and a reduction of the cost for treatment.

### SUMMARY OF THE INVENTION

It is an objective of the present invention to provide a paradigm of treatment for hearing conditions including hearing loss, tinnitus and hyperacusis. It is also an objective to provide a device to aid in the treatment of such hearing conditions singularly or in any combination thereof. It is a further objective of the present invention to provide such a device to treat those conditions with minimal burden on the patient and in a cost effective manner. These objectives are achieved with this device and related diagnosis and/or treatment methods of this present invention. The present invention provides a hearing assistance device that can be programmed to filter, regulate or otherwise manage sounds passing to the patient using an amplifier and a sound generator. It can be used to diagnose and/or treat one or more hearing conditions. The device includes programming to manage sound amplification and sound generation, individually or in combination, tailored to address one or more of hearing loss, tinnitus and hyperacusis. The device enables a paradigm of treatment, referred to herein as a method or process, for treating hearing problems including hearing loss, tinnitus and hyperacusis.

The amplifier component of the device is configured to amplify sounds reaching the ear. The sound generator component of the device is configured to generate selectable sounds that will be referred to herein as “white noise.” The white noise generated may be selectable, variable and customizable for the individual patient. White noise is used herein to mean “dense” white noise, which is generally understood to be a heterogeneous mixture of sound waves over a wide frequency range as well as “contextual” white noise, which are particular noises that evoke certain visualizations, including, but not limited to, the sounds of crashing waves, babbling brooks, wind through the trees, crickets chirping or the like. The method includes an assessment of the patient, ear-level testing of the patient and, if it is determined to be necessary, introduction of the device and the implementation of conditions for its use.

The device is configured to allow the healthcare provider and the patient with the hearing condition to regulate the level of amplification and/or white noise in a manner tailored to treat that patient's particular condition, whether it is hearing loss, tinnitus, hyperacusis or any combination thereof with a single device. The device enables a level of treatment for any or all such conditions. For example, when the patient suffers from tinnitus and hyperacusis, the tinnitus level of treatment can only be accomplished once the level of hyperacusis of treatment has been resolved. That is, as the hyperacusis resolves (being treated first), the need for treatment of the tinnitus (to be treated next) will be less involved. Likewise, a patient presenting with a level of hearing loss and an unrealized level of tinnitus, may find that the diagnosis and treatment of the tinnitus negates the need for treatment of the hearing loss.

### BRIEF DESCRIPTION OF THE DRAWINGS

FIG. 1 is a simplified side view of an embodiment of the device of the present invention.

FIG. 2 is a simplified top view of the embodiment of the device shown in FIG. 1.

FIG. 3 is a simplified diagram of steps of primary steps of the method of the present invention involving use of the device.

FIG. 4 is a simplified representation of an example of four programming blocks that may be incorporated into a processor of the device of the present invention.

FIG. 5 is a simplified representation of the range of control options available for hearing loss, tinnitus and hyperacusis treatments.

FIG. 6 is a graph showing an example tinnitus treatment threshold representation.

FIG. 7 is a graph showing an example hyperacusis sensitivity representation.

FIG. 8 is a graph showing an example of graduated treatment of a tinnitus condition impacted by a hyperacusis condition.

### DETAILED DESCRIPTION OF THE INVENTION

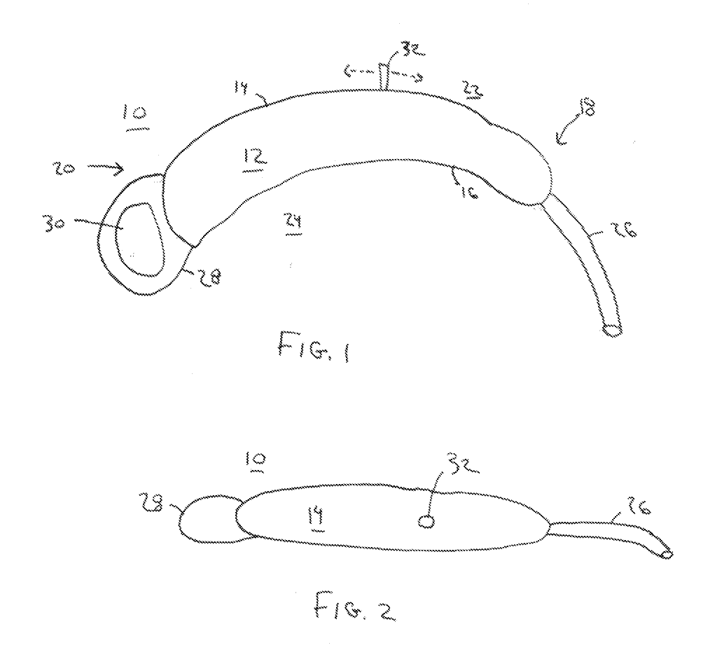

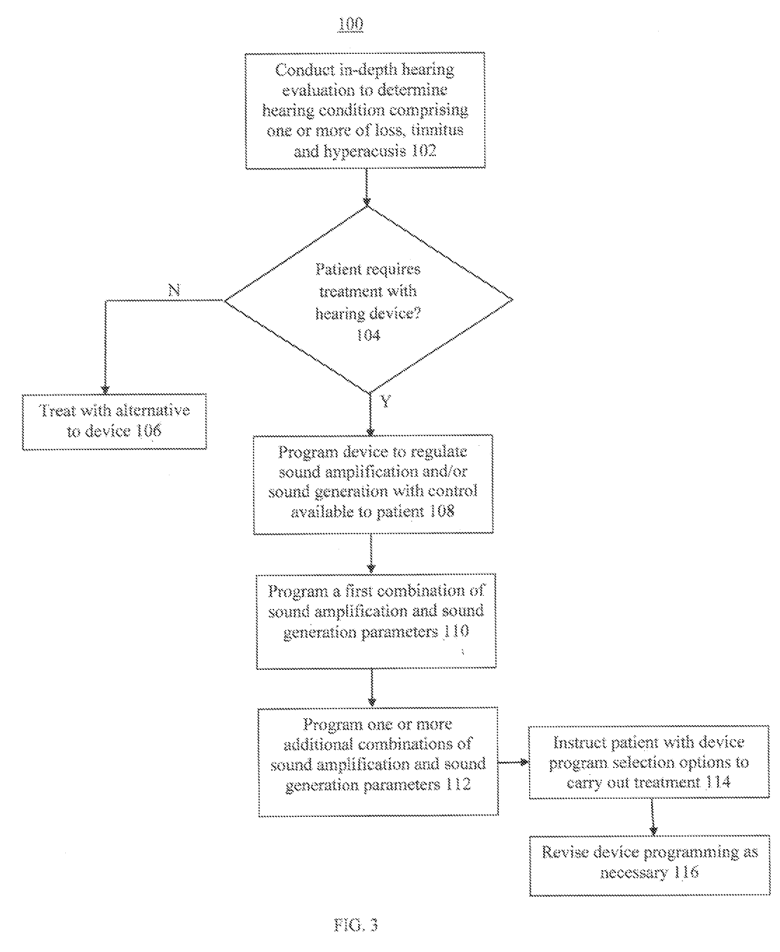

A device 10 suitable for controlling sound delivered to a patient's ear is shown in simplified form in FIGS. 1 and 2. The device 10 includes a main body 12 having an upper portion 14, a lower portion 16, a first end 18 and a second end 20. The main body 12 is of a generally arcuate shape as shown in FIG. 1. The device 10 positioned as shown in FIG. 1 includes a superior portion 22 and an inferior portion 24, wherein the inferior portion 24 makes contact with the ear behind the ear when the device 10 is in use. The first end 18 includes a sound-conducting conduit 26 in the form of a flexible tube that is inserted into the ear and is a sound wave conduit. The second end 20 includes an activation housing 28 for containing a device battery 30 therein. The activation housing 28 is hinged so that the device 10 may be turned on by closing the housing 28 for the battery 30 to make contact with contacts that energize the device 10, and so that the device 10 may be turned off by opening the housing 28. The upper portion 14 includes a controller such as control switch 32 configured to enable selection and adjustment of one or more sound control programs among a plurality of sound control programs contained on a processor located within the main body 12. The sound control programs may be used to regulate operation of either or both of a sound amplifier and a sound generator. The sound amplifier and/or the sound generator may be located within the main body 12 of the device 10 but not limited thereto. Such sound amplifiers and sound generators are of the type generally known to those skilled in the art. The sound amplifier and the sound generator are coupled to the processor such that their operation may be managed by the processor as a function of the programming of the sound control programs and adjustments of sound amplification and/or generation within parameters established as part of the sound control programs. It is to be understood that the controller may be something other than a switch, such as a dial, button or the like that a patient may use to select and/or adjust a sound amplification and/or sound generation program.

By way of simplified representation in FIG. 3, steps of a method 100 of the present invention for treatment of hearing conditions may be performed to carry out such treatment using the device 10. In a first step 102, an in-depth hearing evaluation is conducted on a patient to determine the characteristics of that patient's hearing condition. In second step 104, a determination is made as to whether the patient requires an alternative form of treatment 106 that does not involve the use of a hearing assistance device, or the use of a hearing assistance device 10 of the present invention, step 108. If the device 10 is to be used, step 110 of the method 100 involves the programming of the device 10 with a first combination of sound amplification and sound generation parameters dependent on the diagnosed condition of the patient. Next, one or more additional combinations of sound amplification and sound generation parameters are programmed into the device, step 112. The patient is then provided with programming option information including the parameters of each programmed combination of sound amplification and sound generation, step 114. The patient may then be evaluated upon completion of a treatment regimen comprising the application of one or more of the programmed combinations and the programming may be revised as necessary, step 116.

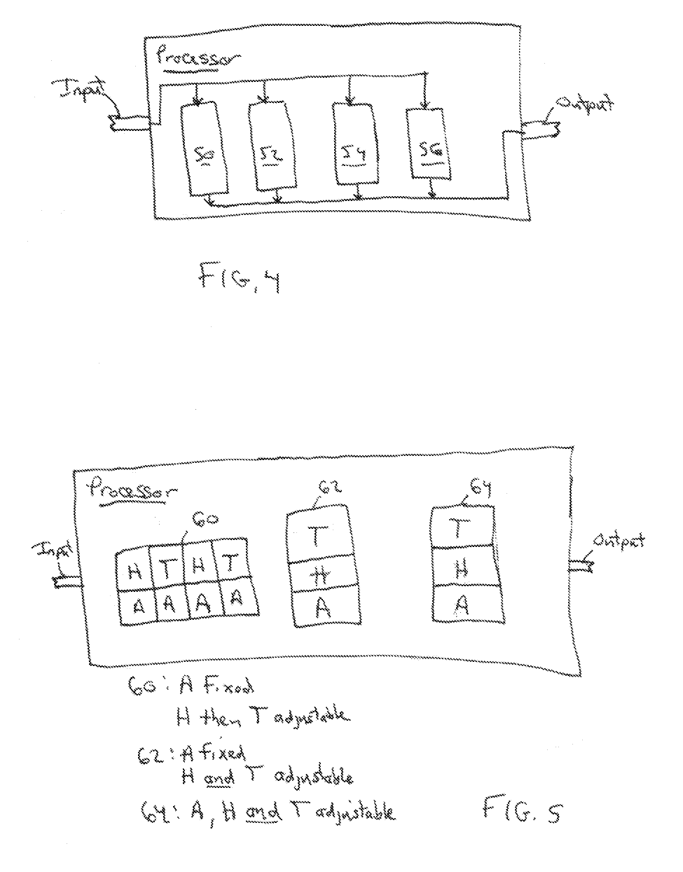

As illustrated in FIG. 4, the processor of the device 10 may be programmed with a plurality of sound amplification and/or sound generation parameters that may be selected by the patient and implemented with the device 10. Although there are four program “blocks” 50, 52, 54 and 56 depicted in FIG. 4, it is to be understood that more or fewer such blocks may be programmed into the device without deviating from the functions of the device as described. The patient may turn the device 10 on by closing the housing 28 containing the battery 30 and thereby either access the last of the program blocks 50-56 used when the device 10 was last on, or access the first program block 50. The patient may change the program block activated by pressing and holding the control switch 32. That is, if program block 50 is first initiated by closing the housing 28, the switch 32 may be pushed and held once to access program block 52, a second time to access program block 54 and a third time to access program block 56. The switch 32 may be pressed and held again to return to program block 50. One or more of the program blocks 50-56 may be configured to enable the patient to adjust either or both of sound amplification and sound generation, dependent on the patient's hearing condition and treatment instructions. Specifically, rather than hold the switch 32 down, the patient may tap and release (“toggle”) the switch 32 toward the first end 18 of the device 10 to increase sound amplification, sound generation amplification or both, dependent on the particular parameters set in the program block selected. The patient may alternatively toggle the switch toward the second end 20 of the device 10 to decrease sound amplification, sound generation or both.

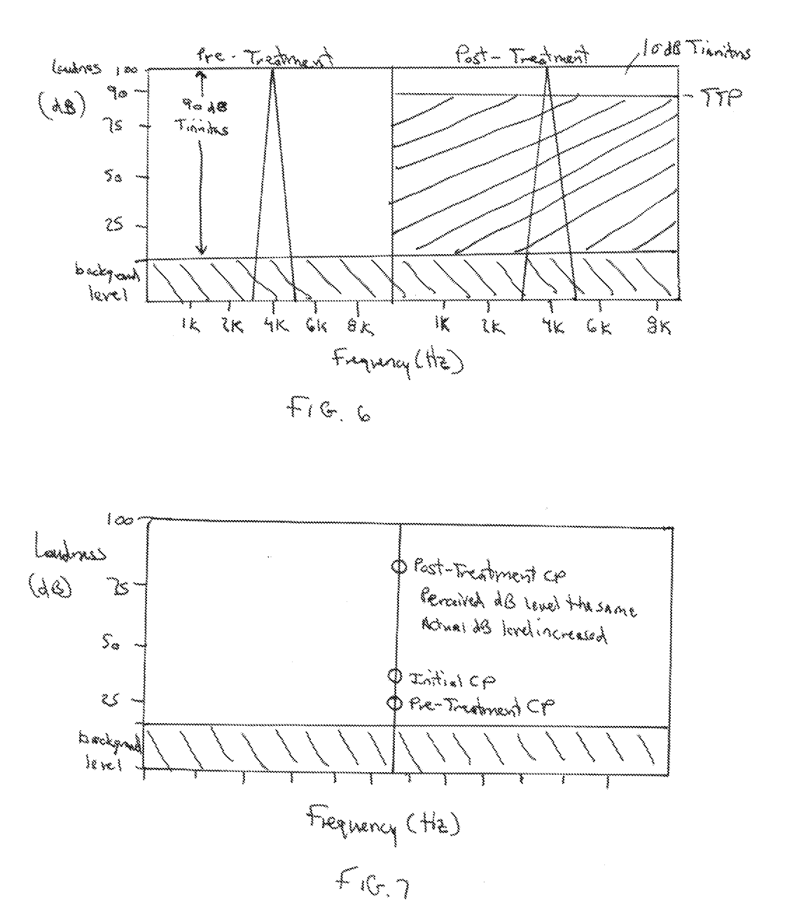

As illustrated in FIG. 5, any of the program blocks 50-56 may comprise selectable programming of sound parameters delivered to the patient's ear through the sound-conducting conduit 26. That is, in a first configuration 60, sound amplification may be fixed by programming and cannot be adjusted by the patient, and hyperacusis or tinnitus treatment sound generation may be adjusted by the patient. In a second configuration 62, sound amplification may be fixed by programming and cannot be adjusted by the patient and hyperacusis and tinnitus treatment generation may both be adjusted by the patient. Finally, in a third configuration 64, sound amplification, hyperacusis treatment and tinnitus treatment sound generation can all be adjusted by the patient. Any of blocks 50-56 may be programmed with ranges of sound amplification and sound generation frequency and amplification associated with configurations 60-64. The ranges may or may not overlap for any given set of program blocks 50-56. Those skilled in the art of hearing condition treatment can instruct a computer programmer to establish sound ranges and upper and lower limits of adjustment permitted by the patient for a given range in a specific program block.

It is to be understood that the programming flexibility enabled with the third configuration 64 provides an efficient mechanism of treatment of hearing loss, tinnitus and hyperacusis. For example, a patient may first be treated for hearing loss by adjusting sound amplification until the patient acknowledges that sound level is sufficient. Next, the sound generator may be programmed to establish a level of loudness sufficient to reach the tinnitus treatment point. In the event the patient also has hyperacusis such that the tinnitus treatment point of loudness exceeds the patient's comfort point for hyperacusis treatment, the third configuration 64 will account for that condition in the operation of the device 10. Specifically, one form of sound may be used to treat the hyperacusis and a second form of sound may be used simultaneously to treat the tinnitus. In order to reach the comfort point associated with hyperacusis treatment, the programming block may be programmed with “dense” white noise available through the sound generator. In addition, the programming block may be programmed with “contextual” white noise available through the sound generator to obtain the tinnitus treatment point. This allows concurrent treatment of hyperacusis and tinnitus with the device 10. Such programming of one or more of the program blocks 50-56 allows a patient to vary all three of hearing loss sound amplification, tinnitus sound generation and hyperacusis sound generation. That programming availability establishes the device 10 as an acoustic modulator.

The sound amplification and sound generation functions described herein using the device 10 may be carried out as electronic functions performed through a computing device such as a programmable processor contained in the main portion 12 of the device 10 and powered by the battery 30 based on computer programming steps. The functions configured to perform the steps and sound generation options described herein may be implemented in hardware and/or software. For example, particular software, firmware, or microcode functions executing on the computing device can provide the sound amplification parameters, the sound generation parameters and the ranges of each permitted to be selected and controlled using the control switch 32. Alternatively, or in addition, hardware modules, such as programmable arrays, can be used in the devices to provide some or all of those functions, provided they are programmed to perform the steps described.

The steps of the programming of the device 10 of the present invention, individually or in combination, may be implemented as a computer program product tangibly as computer-readable signals on a computer-readable medium, for example, a non-volatile recording medium, an integrated circuit memory element, or a combination thereof. Such computer program product may include computer-readable signals tangibly embodied on the computer-readable medium, where such signals define instructions, for example, as part of one or more programs that, as a result of being executed by a computer, instruct the computer to perform one or more processes or acts described herein, and/or various examples, variations and combinations thereof. Such instructions may be written in any of a plurality of programming languages, for example, C++ or any of a variety of combinations thereof. The computer-readable medium on which such instructions are stored may reside on the processor of the device 10 or they may be cached and separately implemented in that processor.

A commercially available product having sound amplification and sound generation availability is the Behind The Ear (BTE) Combo digital aid available from General Hearing Instruments of New Orleans, La. The BTE Combo may be programmed with one or more of the program blocks described herein in a manner known to those capable of making such devices. However, prior to the present invention, no one had thought to modify such a device to allow sound amplification control, sound generation control and program blocks configured to enable the treatment of hearing loss as well as tinnitus and hyperacusis.

The indicator of proper functioning of the device 10 for the treatment paradigm is that the patient feels that he is more comfortable with respect to his hearing condition with the device 10 in place and functioning than he would be without it. This incentivizes the patient to wear the device 10, or any similar device that enables the patient to control sound amplification and/or generation in the ear during all waking hours as they are better with it in place than without it. In most cases, the patient will require two of the devices 10, one in each ear, although it is possible that only one may be used in certain circumstances.

The usual endpoint of treatment with the device 10 is the time when the patient finds that the condition, such as tinnitus, is as good without the device 10 in place as it has been during the treatment course with the device 10 in place. In the case of the patient with tinnitus, for example, the loudness of the tinnitus and the absence of silence secondary to the tinnitus notwithstanding, patients at all levels of severity, gain control over their tinnitus, which is the endpoint of treatment for that condition.

It is noted and as can be concluded from the description of the methods described herein, the device 10 or its equivalent may be used as a hyperacusis only treatment device. It may be used as a tinnitus only treatment device. It may be used as a combination hyperacusis and tinnitus treatment device. It may also be used as a treatment device for hearing loss, either for that condition alone or in combination with either or both of tinnitus and hyperacusis. The device 10 or its equivalent may be used as a tool to test for either or both of tinnitus and hyperacusis. In that regard, it may be used locally with the patient or at a location that is remote from the patient, such as by phone or a computing device.

Specific aspects of tinnitus and hyperacusis (and its related conditions) and treatment thereof enabled with the device of the present invention will now be described.

### Tinnitus Conditions and Treatment Through the Present Invention.

A very consistent “subjective tinnitus patient profile” of what we consider to be the typical “tinnitus patient” has emerged:
1. Patient is obviously and earnestly troubled.
2. Multiple prior medical care provider interactions without meeting patient expectations—many times exacerbating the diagnosis.
3. May be suspicious and find it difficult to develop the necessary trusting relationship.
4. Difficult to describe the problem.
5. No ability to objectivize the problem.
6. No physical evidence of disabling problem noticeable by family, friends or colleagues.
7. Generally a “computer” problem rather than a “vehicular” problem.
8. Unable to find support around their perception that their tinnitus is equal to the “worst” medical diagnoses in terms of decreased productivity and damage to lifestyle.
9. Well incented, easily and effectively engaged and amazingly grateful at any level of positive interaction or perceived benefit.
10. And finally—when all is said:
Unknowingly, desperately attempting to regain “control” over their lives—again.

Current attempts to treat these tinnitus patients—assuming abnormal processing of incoming sound—primarily using medications, counseling and various types of sound therapy—have generally fallen short of these patients' expectations.

### The Treatment Solution of the Present Invention

Demographically, we understand that 95% of any population suffers from some level of tinnitus. Eighty percent of any given population suffers from a level of tinnitus unlikely to be of clinical significance. The remaining 15% of any given population experiences clinically significant tinnitus (CST). Tinnitus patients can be categorized as experiencing level 1, level 2 or level 3 tinnitus. As a level 1 patient, he or she will most likely gain control of his or her tinnitus on a single, first visit and may well require no further visits. As a level 2 patient, further scheduled or unscheduled visits in order to gain control over his or her tinnitus may be necessary. As either a level 1 or level 2 patient, he or she will not be expected to need ear-level therapy in order to accomplish the endpoint of treatment (point at which his or her treatment expectations are realized). As a level 3 patient, there will be a need to extend the treatment beyond that delivered in the first three visits of level 1 and level 2 and more likely than not will require initiation of ear-level instrumentation.

The level 1 and level 2 patients frequently undergo spontaneous resolution—a process which we find is expedited when seen for the first time and requiring no further treatment—clarifying our designation of these patients as non-clinically significant tinnitus patients. The small number of “clinically significant” patients within this group is the acute onset patient who's anxiety level (above average prior to the onset of the tinnitus and exacerbated further by the tinnitus onset) reaches that which disables the patient. In our experience, these are the only level 1 or level 2 patients occasionally requiring ear-level instrumentation.

First Visit—Treatment of Level 1 and Level 2 Patients:

All tinnitus patients (Levels 1-3) are seen as a first visit—60-90 minutes per patient on average. This starts by “listening to the patient”—primarily, but not limited to, an appropriate history and physical. This means at least one hour of a physician's time with each patient—many times longer—developing what we now refer to as the “objective tinnitus patient profile.” This profile determines, and allows us to discuss with the patient, whether there is a need for specialty consultation to further evaluate a potential underlying sinister cause for the tinnitus (pulsatile tinnitus, hypertension, thyroid disorders, Menieres, etc.). Given a negative “objective tinnitus patient profile” we now discuss with the patient the previously mentioned demographics of Tinnitus to allow us to identify for the patient, the level of Tinnitus and the treatment path going forward.

Starting, therefore, with level 1 and level 2 patients—we focus on two treatment components:

1. “Control transfer” (mild)—focuses on anxiety level. This is accomplished in most cases by the treating physician or by the “Tinnitus Life Coach” throughout the course of the first visit. There are many issues of personal control that surface, but the three most common are what we refer to as informational (conversational), reset function (negative to positive) and organizational (sleep deprivation, confusion).
2. Spontaneous habituation follows.
Most often, these infrequent, but treatment deserving level 1 and level 2 patients are quickly identified as having no underlying “sinister” cause for the tinnitus and a level of tinnitus that is minimally bothersome.

Our experience dictates that the anxiety driving this tinnitus is most frequently secondary to a heightened level of anxiety around not knowing the cause of their tinnitus. There are often other mild issues around the control of one's own body and of one's life spheres. With these issues explained (by the Physician or Tinnitus Life Coach) and clearly understood by the patient, the heightened anxiety level resolves and with it the tinnitus (appears to be a linear relationship between anxiety and tinnitus). This may occur literally on-the-spot (during this first appointment) or within a day or two thereafter. These level 1 patients generally need no further treatment.

Level 2 patients are these same level 1 patients who take one or two more appointments to accomplish the same outcome.

Treatment of Level 3 Patients

Level 3 patients are those patients who have had level 1 and level 2 treatment and need further treatment. These patients are the primary focus of the present invention. These are the patients whose tinnitus has not resolved by attempting to decrease their anxiety level and hence the level of the tinnitus (a linear relationship)—as with the level 1 and level 2 patients. These patients need a direct, physical, ear-level control of their tinnitus—an ability to “dial down” the tinnitus—which then decreases the level of anxiety (the same linear relationship approached from the opposite direction). This is accomplished through the use of the device 10 of the present invention, which allows the level 3 patient to do exactly that: “dial down” their tinnitus to a point where it is barely perceptible using one or more of the program blocks of device 10 as described above.

This is accomplished in five steps using the device 10 and modification of its sound amplification and/or sound generation through the programming of the program blocks and instruction to the patient regarding adjustment of sound amplification and/or sound generation using the switch 32.

1. Start the process of setting the Tinnitus Treatment Point (TTP) represented in FIG. 6 by balancing the level of tinnitus, the existing level of hyperacusis and the auditory amplification such that the patient is barely able to perceive the tinnitus. For the TTP example shown in FIG. 6, the left side shows prior to treatment a tinnitus level of 90 decibels and, on the right side, a tinnitus level of only 10 decibels after treatment.
2. Monitor the presence of type B hyperacusis which can be asymptomatic, unrecognized and preclude achieving an effective TTP and may be referred to as “midacusis.”
3. If hyperacusis is present, put tinnitus treatment on hold for 1-2 months while treating hyperacusis with the device 10. As soon as able to achieve the TTP having “reset” the hyperacusis, resume the tinnitus treatment.
4. Maximally focus the TTP by final mixing of the hearing amplification, remaining elements of hyperacusis and the decibel level of the tinnitus.
5. Control transfer (mild)—still continues focusing on the anxiety level. Control transfer (robust)—focuses on the tinnitus (directly and mechanically). It is here that the device 10 of this invention allows the patient to take control of the tinnitus mechanically. This, then, allows treatment of the more profound issues of 1) control of self; 2) self-control; and 3) control of relationships, all of which are at the core of the present treatment paradigm.
In the absence of hyperacusis, steps 1, 2, 4 and 5 may be achieved in a single visit and the patient should, for the first time, be able to control his or her tinnitus. If this level of control matches the patient's expectations as an endpoint of treatment, this is reliably predictive of a successful long term outcome (meeting the patient's expectations) with or without the use of the instrumentation. The patient is now at the end of the treatment paradigm for any of levels 1, 2 and 3.

### Outcomes

The patient is advised at this point of two specific outcomes:

At an average of 24 months, he or she will have accomplished his or her treatment expectations without having to continue wearing the device 10. At the second visit (the fitting of the device 10) the patient is most always able to set his or her tinnitus at a very comfortable, almost imperceptible, level. This level is a level at which the patient's expectations would be met if it could be maintained without continued use of the instrumentation.

A number of patients find that after wearing the device 10 for a period of time, their expectations as to the resolution of the tinnitus have been met—when removed, however, they continue to experience bothersome tinnitus. In this situation the device takes on the same function as eyeglasses in that while wearing them, their expectations of treatment are met. This usually occurs in those with the most severe tinnitus and in the opinion of those individuals, their overall expectation of treatment has been met with or without the instrumentation.

### Conclusions on Tinnitus Treatment

An aspect of rethinking the approach to tinnitus treatment is the concept that we have heard time and again from our patients—a concept which closes the gap in effective management of tinnitus. That concept is that tinnitus is directly related to a level of anxiety which in turn reflects the degree of control that the patient perceives over his or her own body or lifestyle. That concept tells us that there is a “hardwired” physiologic balance between one's level of anxiety, one's level of control of self and one's tinnitus—each affecting the other. This balance can be adjusted to make any one of the three better or worse—in this case, adjusting the amplitude of sound input and minimizing the tinnitus and/or “resetting the hyperacusis.”

An aspect of effective treatment using the device 10 is the consideration of the impact of hyperacusis on tinnitus treatment. Previously, when setting the sound generator component of the device 10, patients would complain of discomfort at low levels of decibel loudness—well below the necessary level to obtain a TTP.

At this point, we were unaware of any consistently effective treatment approach for hyperacusis. But by using a different application of the concept of habituation (from that being used in the TTP), we were able to develop a treatment that has become consistently effective. It was also clear that we were probably dealing with a spectrum of hyperacusis severity rather than a single, more severe level which is more common and more likely to be recognized by the patient or the provider.

Why are we so significantly highlighting the “unrecognized” aspect of hyperacusis? Because we know no one suffers from tinnitus and is unaware of its presence, but it does appear that many may suffer from hyperacusis and be unaware of its presence (and in most cases even its existence). This situation—the presence of hyperacusis, asymptomatic and undiagnosed—led us to proposing a more pragmatic categorization of hyperacusis. This better defined our hyperacusis treatment and allowed a more successful completion of our TTP.

### Hyperacusis Categorization

As with tinnitus, we have based our categorization on there being a spectrum of hyperacusis, differing levels of which respond to differing treatment approaches:

Type A hyperacusis includes both the high decibel and low decibel, symptomatic entities.
Type A, high decibel hyperacusis (classic hyperacusis), appears to be the most frequent and often presents as the primary chief complaint—occasionally, we believe, as a single entity (i.e., without concurrent hearing loss or tinnitus).
Type A, low decibel hyperacusis, which we refer to descriptively as “hypoacusis” is, in our experience, the least common.
Type B, mid decibel hyperacusis may well be the most common, but the least commonly diagnosed (representing the lowest level of awareness among providers).

It is this Type B hyperacusis (which we may refer to as “midacusis”), which necessitated this more detailed categorization. This has allowed us to be more clinically aware of the unrecognized (asymptomatic and undiagnosed) levels of hyperacusis. This also allows for the perception of “hyperacusis” as a spectrum rather than a single level of severity.

### Method of Hyperacusis Treatment

Hyperacusis treatment of the present invention can be best described in a patient with that single diagnosis—no concurrent hearing loss or Tinnitus—a very unusual patient, but a “pure case” example:

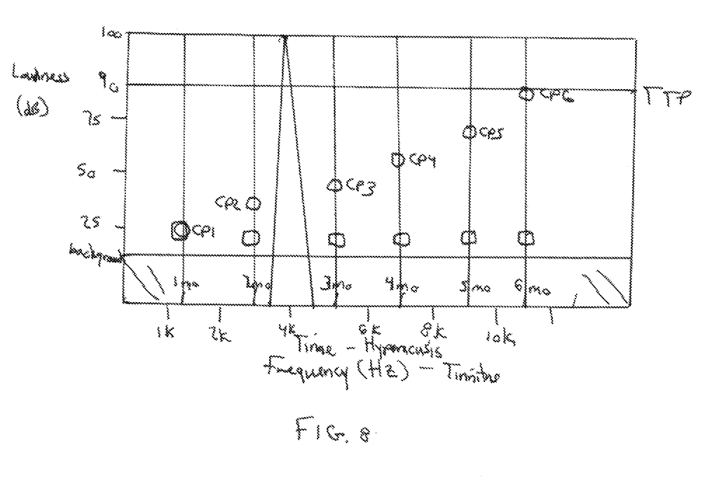

This is done using programmed sound generation of the device 10) in each ear. The volume of the sound generator in the most affected ear is increased slowly focusing totally on the comfort level of that sound. It is important to consider only “comfort” and not “ability to tolerate”. With this “Comfort Point” (CP) identified, the volume is dropped back very slightly to a point where the patient will be comfortable for the full day (12 hours) that the device 10 will be worn. It is this point, represented graphically in FIG. 7 that we identify not only as the CP (for patient identification purposes) but, more importantly, as the Hyperacusis Treatment Point (HTP) for pure treatment purposes.

More specifically, in order to identify the CP, the patient is advised to identify it as a level of comfort (a perceived loudness level) unrelated to the actual level of loudness or any other metric and once this is accomplished, “print it” on his or her brain. This is so that the patient can return to this same CP (actual level of loudness) every morning without regard for the actual decibel level of loudness whether it is louder or softer than the day before. In this step, the patient is setting his or her perceived CP decibel level (at the same perceived decibel level each day) and allowing the actual decibel level to increase to a loudness level that is perceived as unchanged from the starting CP. This focuses the patient on the “actual” comfort and defocuses the “perceived” loudness. After a certain length of time (usually somewhere between 1-10 weeks), the “actual” comfort has not changed (the patient has set it at the same point every day), but the actual loudness is now much higher. The hyperacusis has now been “reset” and the patient is comfortable at a higher decibel level than prior to the treatment.

### Mechanism of Action:

1. For hyperacusis: set perceived decibel level at same perceived level every day—actual decibel level increases, but perceived as the same loudness as the original CP decibel level. Patient expectations are met.
2. For hypoacusis: set actual decibel level (at the same actual loudness every day) and the perceived decibel loudness will decrease. This is essentially a “reverse reset,” which we have found to be beneficial when treating patients, primarily adolescents in our experience.

There appears to be no significant limit to the amount of “reset” that can be achieved although this needs, and we are involved in, further study.

We have developed a very different appreciation of the meaning and significance of hyperacusis (as we have for tinnitus). This is an ongoing process which started with developing The TTP and has led to new levels of understanding of both tinnitus and hyperacusis as well as their interactions with each other and with hearing loss. Hyperacusis exists over a spectrum of severity—the different levels responding to different treatment approaches. All said, present invention effectively addresses hyperacusis at all levels, consistently meeting patient's expectations and enabling the effective treatment of tinnitus in those suffering with hyperacusis by increasing the CP of hyperacusis until such time as the effective TTP can be reached, as represented in FIG. 8. In the example representation of FIG. 8, the desired TIP is shown to be 90 decibels. However, for that patient, the CP of hyperacusis is only 25 decibels. Therefore, the patient cannot reach the TTP at the outset. As a result, over time represented in months on the graphical representation the CP is increased as the perceived level remains the same from CP1 through CP6. The circles represent increasing CP actual decibel level while the squares represent perceived decibel level. When the hyperacusis CP is at or near the TTP at CP6, depicted as taking six months but more or less time may be required, both hyperacusis and tinnitus have been treated with the device 10.

The present invention has been described with respect to various steps and device features. Nevertheless, it is to be understood that various modifications may be made without departing from the spirit and scope of the invention.

What is claimed is:

1. A device for the diagnosis and/or treatment of one or more hearing conditions, the device comprising:
a. a main body including a processor therein;
b. a sound-conducting conduit coupled to the main body;
c. a sound amplifier coupled to the processor;
d. a sound generator coupled to the processor; and
e. a controller coupled to the processor,
wherein the processor is programmed with a plurality of program blocks, wherein each of the plurality of program blocks is programmed to enable selection and/or adjustment of sound amplification and/or sound generation through actuation of the controller and regulation of the sound amplifier and/or the sound generator.

2. The device of claim 1 wherein the controller is a switch that may be actuated to change program block and/or to increase or decrease sound amplification and/or sound generation.
